(19) United States
(12) Patent Application Publication
(10) Pub. No.: US 2008/0097097 A1
(43) Pub. Date: Apr. 24, 2008

(54) Process for Preparing Purine Compounds

(76) Inventor: John A. Ragan, Gales Ferry, CT (US)

Correspondence Address: PFIZER INC., PATENT DEPARTMENT, MS8260-1611, EASTERN POINT ROAD, GROTON, CT, 06340, US

(21) Appl. No.: 11/662,536

(22) PCT Filed: Oct. 10, 2005
(86) PCT No.: PCT/IB05/03255

§ 371 Date: Mar. 8, 2007

Related U.S. Application Data
(60) Provisional application No. 60/621,559, filed on Oct. 22, 2004.

(51) Int. Cl. C07D 473/34

(52) U.S. Cl. 544/277

(57) ABSTRACT

A process for preparing compounds of Formula (I) are described herein as well as key intermediates 1.

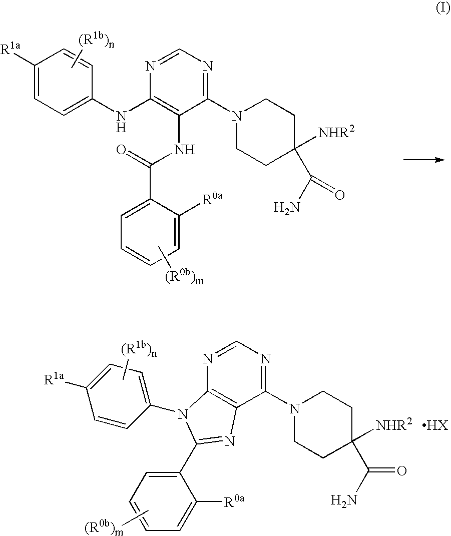

### FIELD OF THE INVENTION

The present invention relates to a process for preparing purine compounds, in particular the preparation of 1-[9-(4-chloro-phenyl)-8-(2-chloro-phenyl)-9H-purin-6-yl]-4-ethylamino-piperidine-4-carboxylic acid amide, and intermediates useful in the synthesis of such purine compounds. The purine compounds prepared by the process described herein have been shown to be CB-1 receptor antagonists.

### BACKGROUND

CB-1 antagonists have been shown to useful for the treatment of a variety of diseases, conditions and/or disorders including obesity, alcoholism, smoking cessation, Parkinson's disease, sexual dysfunctions, dementia, and so forth. Consequently, there exists a desire to develop compounds that antagonize the CB-1 receptor. US Publication No. 2004/0092520 and PCT Publication No. WO 04/037823 describe a series of purine compounds that act as CB-1 antagonists. However, there exists a need to produce purine derivatives, in particular, 1-[9-(4-chloro-phenyl)-8-(2-chloro-phenyl)-9H-purin-6-yl]-4-ethylamino-piperidine-4-carboxylic acid amide, in a more efficient, environmentally safe, and cost effective manner at larger scales of manufacture.

### SUMMARY

The present invention provides an improved process for preparing compounds of Formula (I): 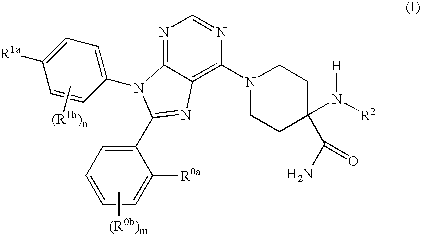
wherein R^{0a}, R^{0b}, R^{1a}, R^{1b} are each selected from the group consisting of chloro, fluoro, (C₁-C₄)alkoxy, (C₁-C₄)alkyl, fluoro-substituted (C₁-C₄)alkyl), and cyano (preferably, R^{0a} and R^{1a} are each chloro, and R^{0b} and R^{1b} are each hydrogen (i.e., n and m are 0)); n and m are each independently 0 or 1 (preferably n and m are 0); and R² is (C₁-C₄)alkyl (preferably, R² is ethyl).

The process for the preparation of the compound of Formula (I) comprises the steps of:

(1) cyclizing the compound of Formula (1g) in the presence of a protic acid to produce a compound of Formula (I-A) 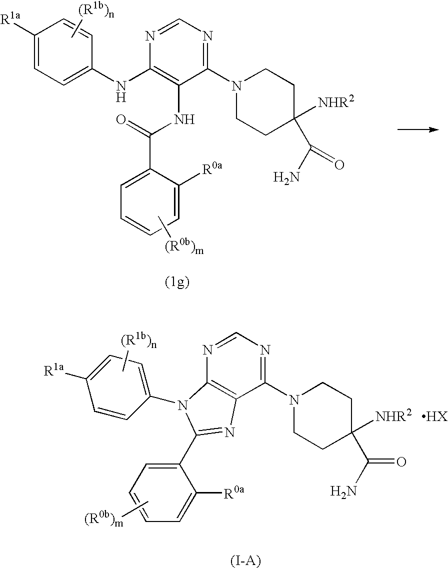
where R^{0a}, R^{0b}, R^{1a}, R^{1b}, R², n and m are as defined for the compound of Formula (I) above, and HX is a protic acid (preferably, the protic acid is hydrochloric acid, sulfuric acid, or phosphoric acid, more preferably, sulfuric acid); and

(2) isolating the compound of Formula (I), a pharmaceutically acceptable salt thereof, or a hydrate or solvate of the compound or the salt.

Preferably, the compound of Formula (I) is isolated as a pharmaceutically acceptable salt selected from the group consisting of hydrochloride, sulfate, phosphate, besylate and mesylate, more preferably, as a hydrochloride or besylate.

The process above may further comprise the step of preparing the compound of Formula (1g) by a process comprising the step of

(a) reacting a compound of Formula (1e) with a compound of Formula (1f) to produce the compound of Formula (1g) 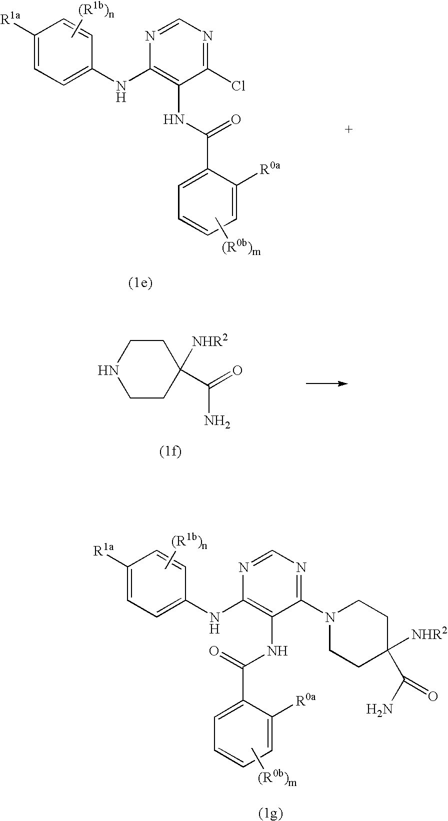
where R^{0a}, R^{0b}, R^{1a}, R^{1b}, R², n and m are as defined for the compound of Formula (I) above. Alternatively, the compound of Formula (1f) may be provided as its corresponding protic acid salt (e.g., hydrochloride, sulfate, phosphate, and the like).

In a preferred embodiment, a process is provided for the preparation of a compound of Formula (IA-1) 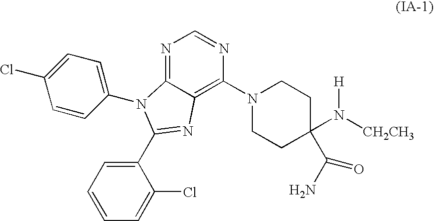
comprising the steps of:

(1) reacting the compound of Formula (I-1e) with a compound of Formula (I-1f) or a protic acid salt thereof to produce a compound of Formula (I-1g)

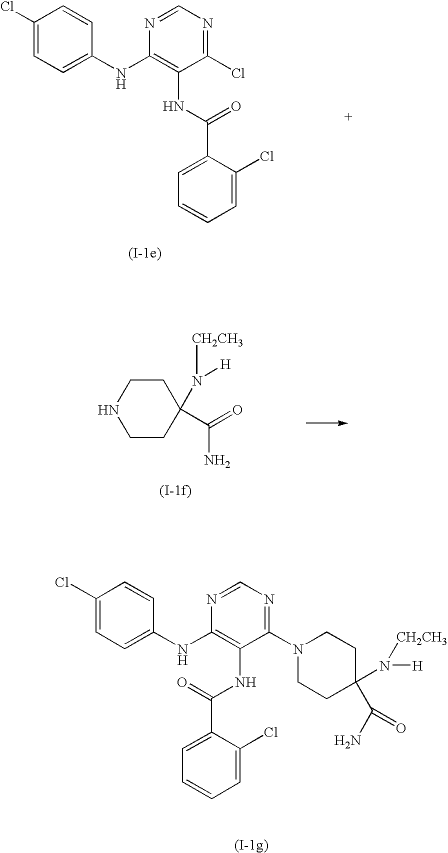

(2) cyclizing the compound of Formula (I-1g) in the presence of a protic acid to produce a compound of Formula (IA-1) 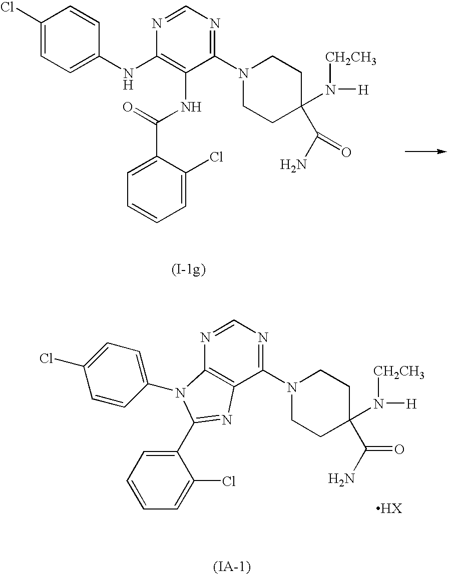
where HX is a protic acid (preferably, the protic acid is selected from the group consisting of hydrochloric acid, methanesulfonic acid, benzensulfonic acid, sulfuric acid, and phosphoric acid, more preferably the protic acid is sulfuric acid); and

(3) isolating the compound of Formula (I), a pharmaceutically acceptable salt thereof or a hydrate or solvate of said compound or said salt.

The isolation step (3) may comprise the steps of (4) converting the protic acid salt (1A-1) to the free base and then (5) optionally converting the free base to a different pharmaceutically acceptable salt. Preferably, the compound of Formula (IA-1) is isolated as a pharmaceutically acceptable salt selected from the group consisting of hydrochloride, sulfate, phosphate, besylate and mesylate, more preferably, as a hydrochloride or besylate.

In another aspect of the present invention, a compound having the Formula (1g) is provided.

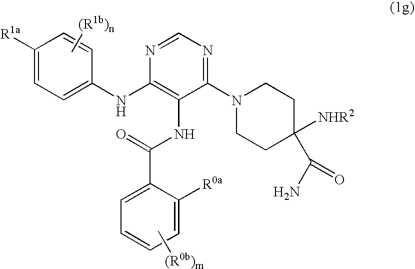

wherein R^{0a}, R^{0b}, R^{1a}, R^{1b} are each independently selected from the group consisting of chloro, fluoro, (C₁-C₄)alkoxy, (C₁-C₄)alkyl, fluoro-substituted (C₁-C₄)alkyl), and cyano; n and m are each independently 0 or 1; and R² is (C₁-C₄)alkyl; or a protic acid salt thereof. Preferably, R^{0a} and R^{1a} are each chloro; n and m are 0; and R² is ethyl.

The process and intermediate described above provides several advantages over the previously described processes. For example, the inventive process is one step shorter than the previously disclosed route (see, US Publication No. 2004/0092520 or PCT Publication No. WO 04/037823) thus providing a more efficient synthesis of the title compounds. Additionally, the inventive process avoids the use of reagents such as phosphorous oxychloride for the preparation of key intermediates. Reagents such as POCl₃ are air- and moisture-sensitive and are therefore difficult to handle on large scale.

### DEFINITIONS

As used herein, the term “protic acid” refers to a compound that donates at least one hydrogen ion (H+) to another compound. Typical protic acids include acids such as hydrochloric acid, hydrobromic acid, sulfuric acid, phosphoric acid, methanesulfonic acid, benzensulfonic acid, acetic acid, and the like.

The term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like).

The term “halo” refers to a chloro, bromo, fluoro or iodo group.

The term “solvate” refers to a molecular complex of a compound represented by Formula (I) and pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The phrase “pharmaceutically acceptable” indicates that the substance or composition must be compatible chemically and/or toxicologically, with the other ingredients comprising a formulation, and/or the mammal being treated therewith.

### DETAILED DESCRIPTION

The starting materials used in the processes described herein are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

In the preparation of the purine compounds, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Scheme I below summarizes the process of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives.

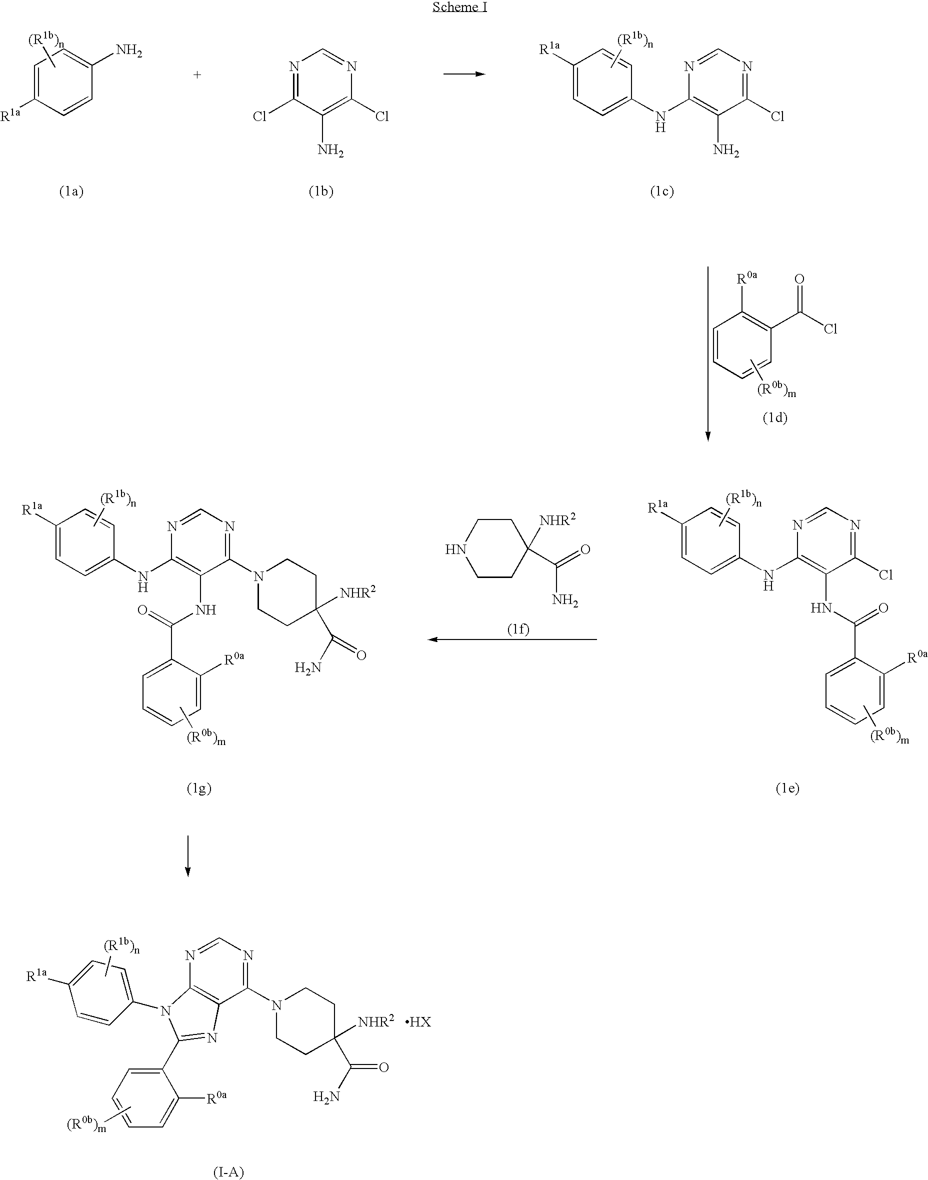

The desired aniline (1a) is coupled with 5-amino-4,6-dichloropyrimidine (1b: available from Aldrich Chemicals, Milwaukee, Wis.) to form intermediate (1c) by suspending the two materials in an acidic aqueous media (e.g., ethanol/water containing a protic acid (e.g. HCl)) followed by heating to an elevated temperature (about 80° C.). The free amino group on intermediate (1c) is then reacted with the desired activated carbonyl compound (1d) to form the amide intermediate (1e). The amidation reaction may be accomplished using procedures well-known to those skilled in the art. For example, intermediate (1c) may be treated with N,N-dimethylacetamide followed by the addition of the desired benzoyl chloride (1d) at a temperature from about 0° C. to about 5° C. The desired 4-alkylaminopiperidine-4-carboxamide compound (1f: see Scheme II below) is then coupled with intermediate (1e) to form intermediate (1d) by reacting the chloride (1e) with the carboxamide (1f) at an elevated temperature (about 80° C.) in the presence of a base (e.g., triethylamine). The carboxamide (1f) may alternatively be provided as its corresponding protic acid salt. Intermediate (1g) is then cyclized to form the protonated compound of Formula (I) (e.g., a compound of Formula (I-A) by heating intermediate (1g) at an elevated temperature (e.g., about 80° C.) in a protic solvent (e.g., isopropanol) in the presence of the desired protic acid (e.g., sulfuric acid, phosphoric acid, or hydrochloric acid). The protonated compound (I-A) may be converted to the free base by neutralizing the acid with a weak base (e.g., Na₂CO₃). If desired, the free base may be reacted with a desired inorganic or organic acid to form a pharmaceutically acceptable salt (e.g., mesylate, besylate and hydrochloride salt).

The preparation of 4-alkylaminopiperidine-4-carboxamide compounds of Formula (1f) is depicted below.

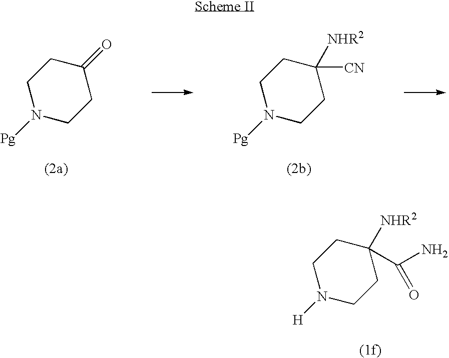

The amino group of 4-piperidinone is first protected to provide intermediate (2a). A useful protection group is benzyl. 4-piperidinone and derivatives thereof may be purchased commercially from a variety of sources (e.g., Interchem Corporation, Paramus, N.J. and Sigma-Aldrich Co., St. Louis, Mo.). Piperidinone (2a) is then reacted with the desired alkylamine and potassium cyanide in an aqueous HCl/ethanol solvent mixture at about 0-30° C. The cyano group is converted to the corresponding amide with acid and water. The protecting group is then removed using conventional methods for the particular protecting group employed. For example, a benzyl protecting group may be removed by hydrogenation in the presence of Pd/C.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography), recrystallization, and differential (i.e., liquid-liquid) extraction techniques.

The compounds may be isolated and used per se or in the form of its pharmaceutically acceptable salt, solvate and/or hydrate. In some instances, the free base is preferred. As used herein the term “free base” refers to an amino group having a lone pair of electrons. The term “salts” refers to inorganic and organic salts of a compound which may be incorporated into the molecule via an ionic bond or as a complex. These salts can be prepared in situ during the final isolation and purification of a compound, or by separately reacting the compound or prodrug with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulfonate salts, and the like. Preferred salts include hydrochloride, mesylate and besylate salts. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The compounds (including intermediates) may contain asymmetric or chiral centers; therefore, the compounds and intermediates may exist in different stereoisomeric forms (e.g., enantiomers and diasteroisomers). It is intended that all stereoisomeric forms of the intermediates and compounds as well as mixtures thereof, including racemic mixtures, form a part of the present invention.

The compounds prepared by the inventive process may exist in unsolvated as well as solvated forms with pharmaceutically acceptable solvents such as water, ethanol, and the like, and it is intended that the invention embrace both solvated and unsolvated forms of the compounds.

It is also possible that the intermediates and compounds may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

The present invention also embraces the use of isotopically-labeled compounds (including intermediates) which are identical to those recited herein, but for the fact that one or more atoms are replaced by an atom having an atomic mass or mass number different from the atomic mass or mass number usually found in nature. Examples of isotopes that can be incorporated into the intermediates or compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds can generally be prepared by following procedures analogous to those disclosed in the Schemes and/or in the Examples herein below, by substituting an isotopically labeled reagent for a non-isotopically labeled reagent.

Compounds made by the process of the present invention are useful for treating diseases, conditions and disorders modulated by cannabinoid receptor antagonists.

Preliminary investigations have indicated that the following diseases, conditions, and/or disorders are modulated by cannabinoid receptor antagonists: eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimer's disease, dementia of aging, vascular dementia, mild cognitive impairment, age-related cognitive decline, and mild neurocognitive disorder), sexual dysfunction in males (e.g., erectile difficulty), seizure disorders, epilepsy, inflammation, gastrointestinal disorders (e.g., dysfunction of gastrointestinal motility or intestinal propulsion), attention deficit disorder (ADD including attention deficit hyperactivity disorder (ADHD)), Parkinson's disease, and type II diabetes.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 or 500 (available from Varian Inc., Palo Alto, Calif.) at room temperature at 400 and 500 MHz ¹H, respectively. Chemical shifts are expressed in parts per million (6) relative to residual solvent as an internal reference. The peak shapes are denoted as follows: s, singlet; d, doublet; t, triplet; q, quartet; m, multiplet; br s, broad singlet; v br s, very broad singlet; br m, broad multiplet; 2s, two singlets. In some cases only representative ¹H NMR peaks are given.

Mass spectra were recorded by direct flow analysis using positive and negative atmospheric pressure chemical ionization (APcl) scan modes. A Waters APcl/MS model ZMD mass spectrometer equipped with Gilson 215 liquid handling system was used to carry out the experiments.

Mass spectrometry analysis was also obtained by RP-HPLC gradient method for chromatographic separation. Molecular weight identification was recorded by positive and negative electrospray ionization (ESI) scan modes. A Waters/Micromass ESI/MS model ZMD or LCZ mass spectrometer equipped with Gilson 215 liquid handling system and HP 1100 DAD was used to carry out the experiments.

Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and only the lower mass ion is given. MS peaks are reported for all examples.

Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Biotage™ columns (ISC, Inc., Shelton, Conn.) under low nitrogen pressure. Radial chromatography was performed using a Chromatotron™ (Harrison Research).

### Starting Materials

Each of the following starting materials may be purchased from Sigma-Aldrich Company (Milwaukee, Wis. USA)

4-Chloroaniline (I-1a)

5-Amino-4,6-dichloropyrimidine (I-1b)

2-Chlorobenzoyl chloride (I-1d)

The preparation of starting material I-1f is described in US Publication No. 2004/0092520 or PCT Publication No. WO 04/037823) and reproduced below.

### Preparation of Starting Material 4-Ethylaminopiperidine-4-carboxylic Acid Amide (I-1f)

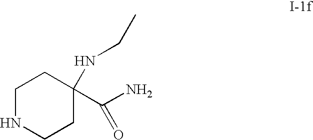

To a solution of 4-N-benzylpiperidone (5.69 g, 29.5 mmol) in ethanol (4.2 ml) cooled in an ice bath was added ethylamine hydrochloride (2.69 g, 32.3 mmol) in water (3 ml) while keeping the internal temperature of the reaction below 10° C. A solution of KCN (2.04 g, 31.3 mmol) in water (7 ml) was added to the reaction solution over 10 minutes while keeping the internal temperature below 10° C. The reaction was then warmed to room temperature and stirred 18 hours. Isopropanol (10 ml) was added to the reaction mixture to give two distinct layers: a lower colorless aqueous layer and an orange organic upper layer. The organic layer was separated and stirred with water (30 ml) for 30 minutes. The organic layer was separated (the orange organic layer became the bottom layer) and the orange oil was diluted in CH₂Cl₂ (30 ml). The organic layer was washed with brine, dried (Na₂SO₄), filtered and concentrated, in vacuo, to give 1-benzyl-4-ethylaminopiperidine-4-carbonitrile as an orange oil (6.05 g, 84%): +APCl MS (M+1) 244.2; ¹H NMR (400 MHz, CD₂Cl₂) δ 7.32 (d, J=4.1 Hz, 4H), 7.29-7.23 (m, 1H), 3.54 (s, 2H), 2.81-2.76 (m, 2H), 2.75 (q, J=7.1 Hz, 2H), 2.35-2.29 (m, 2H), 2.01-1.98 (m, 2H), 1.74-1.68 (m, 2H), 1.14 (t, J=7.1 Hz, 3H).

A solution of 1-benzyl-4-ethylaminopiperidine-4-carbonitrile (0.58 g, 2.38 mmol) in methylene chloride (2 ml) cooled in an ice bath was treated with H₂SO₄ (1.8 ml, 33 mmol), dropwise, while keeping the internal temperature below 20° C. The reaction was then warmed to room temperature and stirred for 19 hr. After stirring was discontinued, the thick pale orange H₂SO₄ bottom layer was separated, cooled in an ice bath and then carefully quenched with concentrated NH₄OH while keeping the internal temperature below 55° C. The aqueous layer was extracted with methylene chloride (2×10 ml), the combined organic layers were washed with brine (20 ml), dried (Na₂SO₄), and then concentrated in vacuo to afford 1-benzyl-4-ethylaminopiperidine-4-carboxylic acid amide as a pale orange oil that solidified to a peach colored solid upon standing (0.54 g, 87%): +APCl MS (M+1) 262.2; ¹H NMR (400 MHz, CD₂Cl₂) δ 7.34-7.30 (m, 4H), 7.29-7.21 (m, 1H), 7.16 (br s, 1H), 3.48 (s, 2H), 2.71-2.68 (m, 2H), 2.47 (q, J=7.0 Hz, 2H), 2.17-2.02 (m, 4H), 1.62-1.58 (m, 2H), 1.41 (br s, 1H), 1.09 (t, J=7.0 Hz, 3H).

To a solution of 1-benzyl-4-ethylaminopiperidine-4-carboxylic acid amide (7.39 g, 28.3 mmol) in methanol (100 ml) was added 20% Pd(OH)₂ on carbon (50% water; 1.48 g). The mixture was placed on a Parr® shaker and was reduced (50 psi H₂) at room temperature overnight. The mixture was filtered through a pad of Celite®, and then concentrated to a colorless solid I-1f (4.84 g, quantitative): +APCl MS (M+1) 172.2; ¹H NMR (400 MHz, CD₂Cl₂) δ 2.89 (ddd, J=12.9, 8.7, 3.3 Hz, 2H), 2.75 (ddd, J=12.9, 6.6, 3.7 Hz, 2H), 2.45 (q, J=7.2 Hz, 2H), 1.95 (ddd, J=13.7, 8.3, 3.7 Hz, 2H), 1.55 (ddd, J=13.7, 6.6, 3.3 Hz, 2 h), 1.08 (t, J=7.1 Hz, 3H).

### Preparation of Key Intermediate

### Preparation of Intermediate 6-Chloro-N4-(4-chlorophenyl)-pyrimidine-4,5-diamine (I-1c)

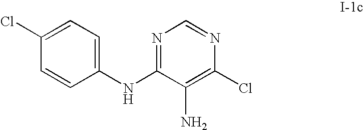

5-amino-4,6-dichloropyrimidine (5.00 g, 29 mmol) and 4-chloroaniline (4.71 g, 36 mmol) were suspended in 80 ml H₂O and 12 ml ethanol. Concentrated HCl (1.2 ml, 14.5 mmol) was added at room temperature followed by warming the reaction to 82° C. After stirring for 19 hours the reaction was cooled to room temperature and stirred for 60 hours. The precipitate was collected on a sintered glass funnel and rinsed with water followed by hexanes. After drying under vacuum, I-1c was obtained as an off-white solid (7.38 g, 98%): +ESI MS (M+1) 255.3; ¹H NMR: (400 MHz, CD₃OD): δ 7.87 (s, 1H), 7.66 (d, J=8.7 Hz, 2H), 7.30 (d, J=8.7 Hz, 2H).

### Example 1

### Preparation of 2-Chloro-N-[4-chloro-6-(4-chlorophenylamino)-pyrimidin-5-yl]-benzamide (I-1e)

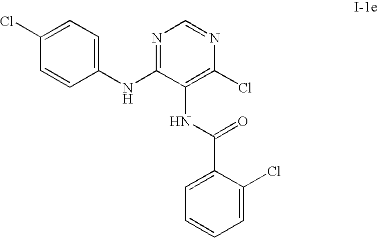

6-Chloro-N4-(4-chlorophenyl)-pyrimidine-4,5-diamine I-1c (1.00 g, 3.92 mmol) was dissolved in 6 ml of N,N-dimethylacetamide giving a clear brown solution. After cooling to 5° C., neat 2-chlorobenzoyl chloride (0.80 g, 4.34 mmol) was added over 1 minute. The solution was warmed to room temperature and stirred for 4 hours. Addition of water (15 ml) caused a white precipitate to come out of solution. The mixture was stirred for an additional 30 minutes at room temperature, then the precipitate was collected by vacuum filtration and rinsed with H₂O followed by hexanes. The solid was further dried under vacuum to give I-1e as a colorless solid (1.27 g, 82%): +APCl MS (M+1) 393.1; ¹H NMR (400 MHz, DMSO-d₆) δ 10.02 (s, 1H), 9.11 (s, 1H), 8.40 (s, 1H), 7.93 (dd, J=7.4, 1.6 Hz, 1H), 7.66-7.40 (m, 7H).

### Preparation of 1-[5-(2-Chloro-benzoylamino)-6-(4-chloro-phenylamino)-pyrimidin-4-yl]-4-ethylamino-piperidine-4-carboxylic acid amide (I-1q)

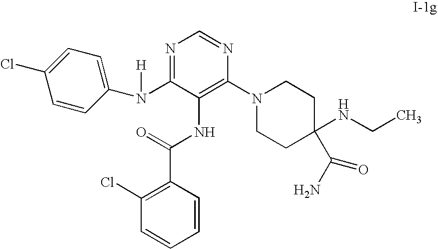

1-[5-(2-Chloro-benzoylamino)-6-(4-chloro-phenylamino)-pyrimidin-4-yl]-chloride (I-1e) (1.10 g, 2.79 mmol), piperidine (I-1f) (0.72 g, 4.2 mmol, 1.5 equiv), triethylamine (0.58 ml, 4.2 mmol, 1.5 equiv), and isopropanol (11 ml) were combined and placed in an 80° C. oil bath. The reaction was monitored by TLC, HPLC and/or mass specpectrometry. After 20 hours, the reaction mixture was cooled and transferred dropwise into 50 ml of ice water. The resulting solids were stirred and granulated at 0° C. to room temperature for 72 hours, then collected by filtration and rinsed with cold water. The product I-1q was isolated as a white to off-white solid (1.51 g, 2.8 mmol, quantitative yield).

¹H NMR (CDCl₃): δ 8.34 (1H, s), 8.02 (1H, s), 7.73 (1H, s), 7.70-7.67 (1H, m), 7.50-7.38 (5H, m), 7.32-7.23 (3H, m), 5.41 (1H, d, J=5), 3.56-3.51 (2H, m), 3.18-3.11 (2H, m), 2.47 (2H, q, J=7), 2.13-2.06 (2H, m), 1.71 (2H, br s), 1.08 (3H, t, J=7). Mass Spec (chemical ionization): 528

### Preparation of 1-[9-(4-Chloro-phenyl)-8-(2-chloro-Phenyl)-9H-purin-6-yl]-4-ethylamino-piperidine-4-carboxylic acid amide (1A-1)

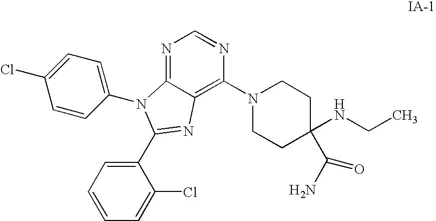

1-[5-(2-Chloro-benzoylamino)-6-(4-chloro-phenylamino)-pyrimidin-4-yl]-4-ethylamino-piperidine-4-carboxylic acid amide (I-1g) (1.48 g, 2.80 mmol) and isopropanol (15 ml) were combined and stirred at room temperature. Concentrated H₂SO₄ (0.47 ml, 8.4 mmol) was added, and the reaction was placed in an 80° C. oil bath. Reaction progression was monitored by HPLC, TLC or mass spectrometry. After 23 hours, the reaction was allowed to cool to room temperature, filtered and rinsed with cold isopropanol. The hydrogensulfate salt of 1A-1 was isolated as a white to off-white solid (1.67 g, 2.74 mmol, 98% yield).

¹H NMR (DMSO-d₆): δ 8.79 (2H, br s), 8.33 (1H, s), 8.04 (1H, s), 7.89 (1H, s), 7.70 (1H, dd, J=7, 2), 7.52-7.44 (5H, m), 7.33-7.30 (2H, m), 4.4 (2H, br s), 3.9 (2H, br s), 2.90-2.89 (2H, m), 2.37 (2H, m), 1.95 (2H, m), 1.21 (3H, t, J=7). Mass Spec (chemical ionization): 510.

Conversion of 1-[9-(4-Chloro-phenyl)-8-(2-chloro-phenyl)-9H-purin-6-yl]-4-ethylamino-piperidine-4-carboxylic acid amide hydrogensulfate salt to free base (1A-1): 1-[9-(4-Chloro-phenyl)-8-(2-chloro-phenyl)-9H-purin-6-yl]-4-ethylamino-piperidine-4-carboxylic acid amide hydrogensulfate salt (1.655 g, 2.72 mmol) was slurried in 17 ml H₂O and 8.5 ml acetone, and treated with Na₂CO₃ (0.317 g, 2.99 mmol). The resulting slurry was placed in a 50° C. oil bath for 90 min, then allowed to cool to room temperature for a period of 2 hours. The resulting solids were collected by filtration, rinsed with cold water and then dried in a vacuum oven to provide free base 1-[9-(4-chloro-phenyl)-8-(2-chloro-phenyl)-9H-purin-6-yl]-4-ethylamino-piperidine-4-carboxylic acid amide (1A-1) as a white solid (1.148 g, 2.25 mmol, 83% yield).

¹H NMR in CDCl₃ (ppm) δ 7.53-7.50 (m, 1H), 7.38-7.33 (m, 3H), 7.24-7.21 (m, 2H), 7.16-7.13 (m, 2H), 4.45 (br s, 2H), 4.02 (t, 2H), 3.90 (br s, 2H), 1.69 (t, 3H); ms (LCMS) m/z=452.2 (M+1). Combustion analysis was calculated for C₂₅H₂₅N₇OCl₂: 55.77%; H, 3.79%; N, 9.29%. Found: C, 55.69%; H, 3.52%; N, 9.13%.

Conversion to HCl salt: 1-[9-(4-Chloro-phenyl)-8-(2-chloro-phenyl)-9H-purin-6-yl]-4-ethylamino-piperidine-4-carboxylic acid amide 1A-1 (1.13 g, 2.21 mmol) was slurried in 17 ml tetrahydrofuran and warmed to 50° C. Concentrated HCl (0.20 ml, 2.43 mmol) was added, and the oil bath temperature increased to 70° C. After 3 hours, the slurry was cooled to room temperature, and stirred overnight. The product was isolated by filtration, rinsed with isopropanol, and air-dried to provide the HCl salt as a white solid (1.30 g, 107% of theory due to residual solvent). Spectral properties were identical to those reported previously.

What is claimed is:

1. A process for preparing a compound of Formula (I): 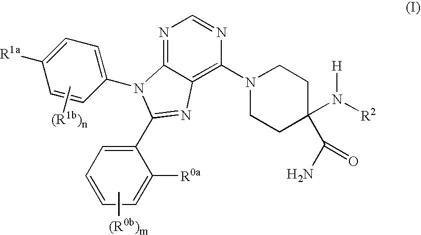
wherein R^{1a}, R^{0b}, R^{1a}, R^{1b} are each independently selected from the group consisting of chloro, fluoro, (C₁-C₄)alkoxy, (C₁-C₄)alkyl, fluoro-substituted (C₁-C₄)alkyl), and cyano; n and m are each independently 0 or 1; and R² is (C₁-C₄)alkyl;
comprising the steps of:
(1) cyclizing a compound of Formula (1g) in the presence of a protic acid to produce a compound of Formula (I-A) 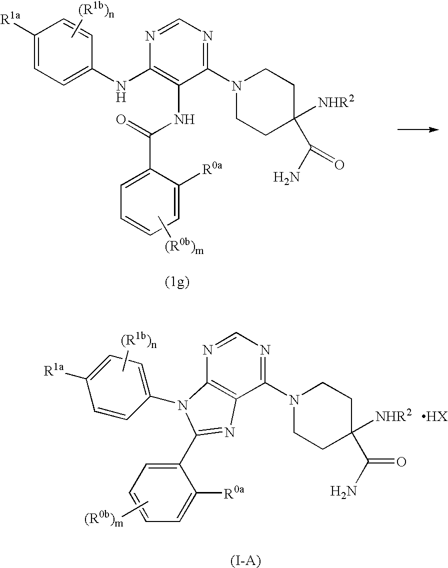
where R^{0a}, R^{0b}, R^{1a}, R^{1b}, R², n and m are as defined for the compound of Formula (1) above, and HX is a protic acid; and
(2) isolating the compound of Formula (I), a pharmaceutically acceptable salt thereof or a hydrate or solvate of said compound or said salt.

2. The process of claim 1 wherein the isolation step (3) comprises the steps of
(4) converting said compound of Formula (I-A) to its corresponding free base; and
(5) optionally converting said free base to its pharmaceutically acceptable salt.

3. The process of claim 1 further comprising the step of preparing said compound of Formula (1g) by a method comprising the step of
(a) reacting a compound of Formula (1e) with a compound of Formula (1f) or a protic acid salt thereof to produce said compound of Formula (1g) 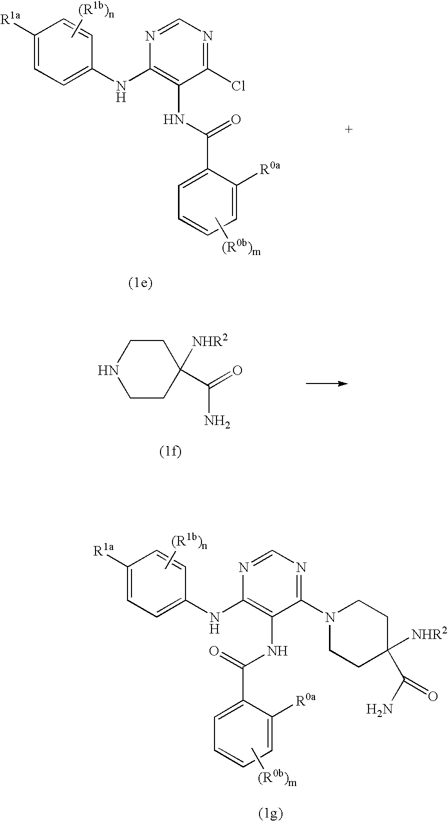
where R^{0a}, R^{0b}, R^{1a}, R^{1b} are each independently selected from the group consisting of chloro, fluoro, (C₁-C₄)alkoxy, (C₁-C₄)alkyl, fluoro-substituted (C₁-C₄)alkyl), and cyano; n and m are each independently 0 or 1; and R² is (C₁-C₄)alkyl.

4. The process of claim 1 wherein said protic acid, HX, is selected from the group consisting of hydrochloric acid, methanesulfonic acid, benzenesulfonic acid, sulfuric acid, and phosphoric acid.

5. The process of claim 8 wherein said protic acid is sulfuric acid.

6. A process for preparing a compound of Formula (IA-1): 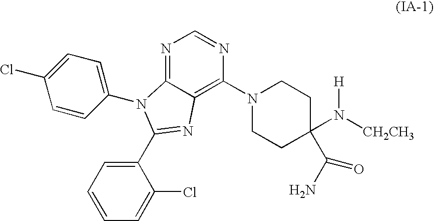
comprising the steps of:
(1) reacting the compound of Formula (I-1e) with a compound of Formula (I-1f) or a protic acid salt thereof to produce a compound of Formula (I-1g) 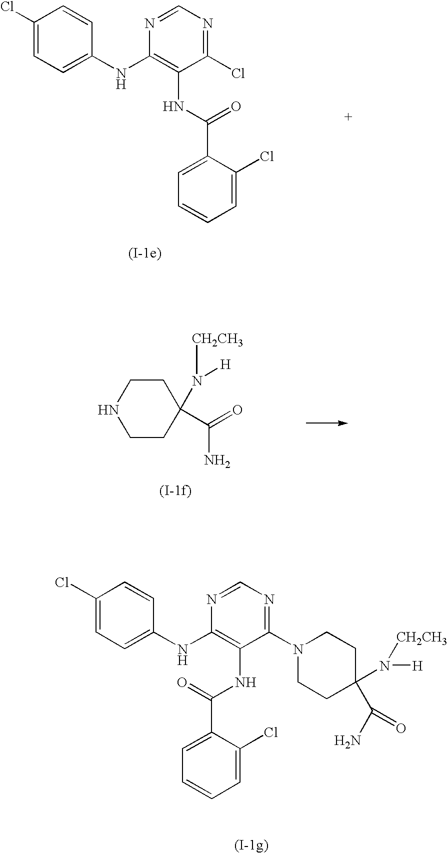
(2) cyclizing the compound of Formula (I-1g) in the presence of a protic acid to produce a compound of Formula (IA-1) 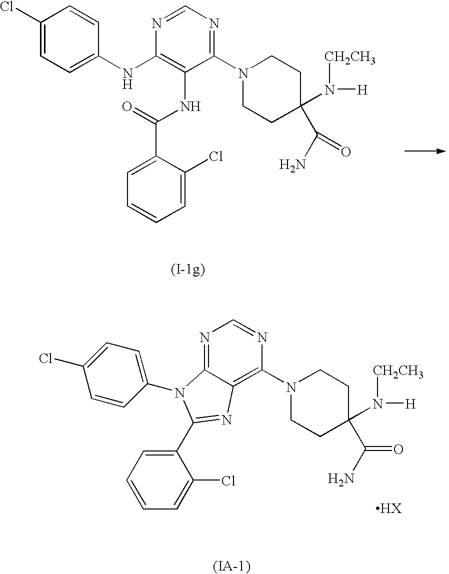
where HX is a protic acid; and
(3) isolating the compound of Formula (I), a pharmaceutically acceptable salt thereof or a hydrate or solvate of said compound or said salt.

7. The process of claim 6 wherein said isolation step (3) comprises the steps of
(4) converting said compound of Formula (I-A) to its corresponding free base; and
(5) optionally converting said free base to its pharmaceutically acceptable salt.

8. The process of claim 7 wherein said compound of Formula (IA-1) is isolated as a pharmaceutically acceptable salt selected from the group consisting of hydrochloride, sulfate, phosphate, besylate and mesylate.

9. The process of claim 8 wherein said pharmaceutically acceptable salt is hydrochloride.

10. The process of claim 8 wherein said pharmaceutically acceptable salt is besylate.

11. A compound having the Formula (1g) 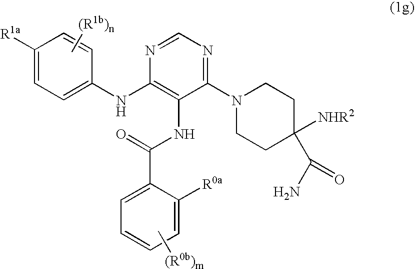
wherein R^{0a}, R^{0b}, R^{1a}, R^{1b} are each independently selected from the group consisting of chloro, fluoro, (C₁-C₄)alkoxy, (C₁-C₄)alkyl, fluoro-substituted (C₁-C₄)alkyl), and cyano; n and m are each independently 0 or 1; and R² is (C₁-C₄)alkyl;
or a protic acid salt thereof.

12. The compound of claim 11 where R^{0a} and R^{1a} are each chloro; n and m are 0; and R² is ethyl.
